(19) United States
(12) Patent Application Publication
(10) Pub. No.: US 2024/0145090 A1
(43) Pub. Date: May 2, 2024

(54) MEDICAL LEARNING APPARATUS, MEDICAL LEARNING METHOD, AND MEDICAL INFORMATION PROCESSING SYSTEM

(71) Applicant: CANON MEDICAL SYSTEMS CORPORATION, Tochigi (JP)

(72) Inventor: Yusuke KANO, Nasushiobara (JP)

(73) Assignee: CANON MEDICAL SYSTEMS CORPORATION, Tochigi (JP)

(21) Appl. No.: 18/486,344

(22) Filed: Oct. 13, 2023

Related U.S. Application Data
(60) Provisional application No. 63/421,359, filed on Nov. 1, 2022.

(30) Foreign Application Priority Data
Jun. 6, 2023 (JP) 2023-093260

(51) Int. Cl. G16H 50/20; G06N 5/04

(52) U.S. Cl. CPC G16H 50/20; G06N 5/04

(57) ABSTRACT

A medical learning apparatus acquires a data set consisting of a plurality of events, the data set including first data that includes first action data relating to an expert. The medical learning apparatus trains, based on the first data, a causal structure model for inferring a causal relationship relating to the plurality of events.

### CROSS-REFERENCE TO RELATED APPLICATION(S)

This application is based upon and claims the benefit of priority from U.S. Provisional Application No. 63/421,359, filed Nov. 1, 2022, and Japanese Patent Application No. 2023-093260, filed Jun. 6, 2023, the entire contents of all of which are incorporated herein by reference.

### FIELD

Embodiments described herein relate generally to a medical learning apparatus, a medical learning method, and a medical information processing system.

### BACKGROUND

In the medical field, it is important to determine treatment plans with accurate consideration of causal relationships. Specifying a causal structure as a graphical model (e.g., a directed acyclic graph or DAG) by machine learning is called “causal structure learning” or “causal discovery”. Using an accurate causal structure leads to improvement in accuracy in downstream tasks, such as diagnosis of disease, individualized treatment effect prediction, dynamic treatment regimens, etc. With a technique of learning a causal structure from non-intervened data, such as randomized comparison test, etc., a causal structure is specified through introducing causal identifiability conditions, such as conditional independency and information criterion, etc.; however, estimation beyond estimation using a Markov equivalence class cannot be achieved except for in special cases. It is therefore difficult to specify a causal structure from observation data.

### BRIEF DESCRIPTION OF THE DRAWINGS

FIG. 1 is a diagram showing an example of a network structure of a medical learning system according to an embodiment.

FIG. 2 is a diagram showing a data structure of a data sample relating to a medical event.

FIG. 3 is a diagram showing a configuration of a medical learning apparatus according to the present embodiment.

FIG. 4 is a diagram showing an example of a network structure of a causal structure model.

FIG. 5 is a diagram showing an example of procedures of medical learning processing.

FIG. 6 is a drawing schematically showing the medical learning processing shown in FIG. 5.

FIG. 7 is a diagram showing a data structure of a data sample according to the medical learning processing shown in FIG. 5.

FIG. 8 is a diagram showing transmission and receipt of data between a causal structure model, a reward function, and a policy function.

FIG. 9 is a diagram showing a data structure of a data sample according to an applied example.

### DETAILED DESCRIPTION

A medical information processing apparatus according to an embodiment is a medical learning apparatus that includes processing circuitry configured to acquire a data set consisting of a plurality of events, the data set including a first data sample that includes first action data relating to an expert, and to train, based on the first data sample, a causal structure model for inferring a causal relationship relating to the plurality of events.

Hereinafter, a medical learning apparatus, a medical learning method, and a medical information processing system according to the present embodiment will be described with reference to the accompanying drawings.

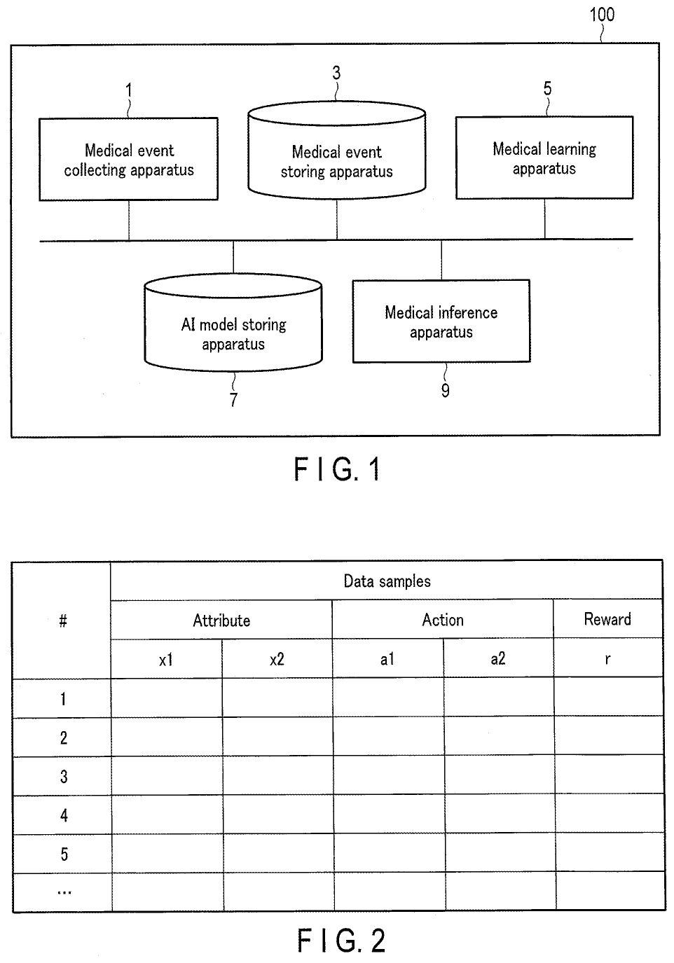

FIG. 1 is a diagram showing an example of a network structure of a medical information processing system 100 according to the present embodiment. As shown in FIG. 1, a medical information processing system 100 includes a medical event collecting apparatus 1, a medical event storing apparatus 3, a medical learning apparatus 5, an AI model storing apparatus 7, and a medical inference apparatus 9. The medical event collecting apparatus 1, the medical event storing apparatus 3, the medical learning apparatus 5, the AI model storing apparatus 7, and the medical inference apparatus 9 are connected by wire or wirelessly to each other in such a manner that they can communicate with each other. The number of each of the medical event collecting apparatus 1, the medical event storing apparatus 3, the medical learning apparatus 5, the AI model storing apparatus 7, and the medical inference apparatus 9 included in the medical information processing system 100 may be one or more.

The medical event collecting apparatus 1 collects data samples relating to medical events. A “medical event” is an event relating to medical care given to a medical care recipient. A medical care recipient is, for example, a patient. A medical event is defined by an attribute and/or an action.

An attribute is data representing a state of a medical care recipient and/or exposure. Examples of state elements are a blood pressure, a heart rate, a blood glucose level, SpO2, and other biological information. Elements of an exposure are a chemical substance or a physical stimulus that a medical care recipient is exposed to, specifically a name of a chemical substance or a physical stimulus and a length of exposure, etc. Data relating to an attribute is collected by a biological information collecting device selected depending on a type of biological information. An attribute is not only data collected by a biological information collecting device but also a medical image collected by various medical image diagnosis apparatuses and an image measurement value, measured by an image processing apparatus based on the medical image, for example. An attribute may be a result of a medical examination by interview with a medical care recipient conducted by a medical care provider, an X-ray interpretation report, or content of an electronic medical record. An attribute may be represented by a scalar quantity corresponding to one of the various attributive elements or by a vector quantity or a matrix quantity that includes a combination of multiple attributive elements. A value of an attribute may be represented by numbers, letters, symbols, etc. Examples of the medical event collecting apparatus 1 that collects data relating to an attribute include a biometric information collecting device, a medical event collecting apparatus, a medical image processing apparatus, and a computer terminal used by a medical care provider during medical diagnosis and treatment, etc. A medical care provider is a doctor, a nurse, a pharmacist, or a care worker.

An action means an action taken for a medical care recipient having a certain attribute. Specifically, an action is a medical diagnosis and treatment action taken by a medical care provider for a medical care recipient, an action taken by a medical care recipient in response to an instruction from a medical care provider, or an action that a medical care recipient voluntarily takes. Examples of action elements are a medication treatment, a surgical operation, a radiotherapy, etc. An action may be represented by a scalar quantity corresponding to one of the various action elements or a vector quantity or a matrix quantity that includes a combination of a plurality of action elements. A value of an action is represented by numbers, letters, symbols, etc. Examples of the medical event collecting apparatus 1 that collects action data include a computer, etc. used by a medical care provider or a medical care recipient.

A data sample relating to a medical event may include a reward in addition to an attribute and an action. A reward is data to evaluate the action performed for a medical care recipient having the attribute. Reward elements are a clinical outcome, a patient report outcome, an economic outcome, for example. Examples of a clinical outcome include a morbidity rate (including whether a patient is affected by a disease or not), a five-year survival rate (including whether a patient survived or not), a complication rate (including whether or not a patient suffers from a complication), a readmission rate (including whether a patient is re-hospitalized or not), an examination value (or a level of improvement in an examination value), a degree of independence in a patient's daily life, etc. Examples of a patient report outcome include a subjective symptom, a subjectively observed health state, a level of satisfaction toward a treatment, and a subjectively observed happiness level. Examples of an economic outcome include medical bills, committed medical resources, the number of hospitalized days, etc. A reward may be represented by a scalar quantity corresponding to one of the various reward elements or a vector quantity or a matrix quantity that includes a combination of a plurality of reward elements. A value of a reward is represented by numbers, letters, symbols, etc. Examples of the medical event collecting apparatus 1 that collects reward data include a computer terminal, etc. used by a medical care provider or a medical care recipient.

FIG. 2 is a diagram showing a data structure of a data sample relating to a medical event. As shown in FIG. 2, a data sample relating to a medical event includes data of an attribute, an action, and/or a reward. In the present embodiment, an attribute is represented by a symbol “x”, an action is represented by a symbol “a”, and a reward is represented by a symbol “r”. The indices appended to the symbols are numbers for identifying attributive elements, action elements, or reward elements. Although no index is appended to the reward r in the example of FIG. 2, the reward r is appended by an index in a case where a reward is defined by two or more elements.

The medical event storing apparatus 3 is a computer that includes a storage apparatus for storing a data set consisting of data samples relating to medical events. As the storage apparatus, a ROM (read only memory), a RAM (random access memory), an HDD (hard disk drive), an SSD (solid state drive), or an integrated circuit storage device, etc. storing various types of information may be used.

The medical learning apparatus 5 is a computer for training a causal structure model for inferring a causal relationship with respect to a plurality of medical events. The details of the medical learning apparatus 5 are described later.

The AI model storing apparatus 7 is a computer that includes a storage apparatus for storing a causal structure model, etc. trained by the medical learning apparatus 5. As the storage apparatus, a ROM, a RAM, an HDD, an SSD, or an integrated circuit storage apparatus may be used.

The medical inference apparatus 9 is a computer for inferring a causal relationship between a plurality of medical events using a trained causal structure model.

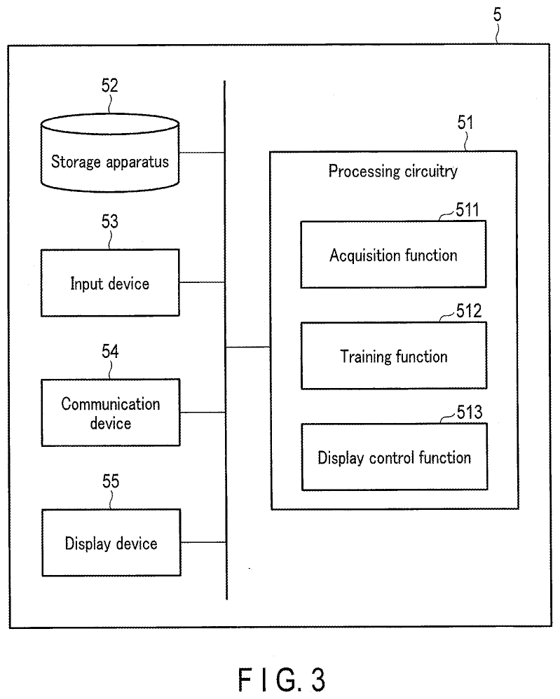

FIG. 3 is a diagram showing a configuration example of the medical learning apparatus 5. As shown in FIG. 3, the medical learning apparatus 5 is an information processing device, such as a computer having processing circuitry 51, a storage apparatus 52, an input device 53, a communication device 54, and a display device 55. The processing circuitry 51, the storage apparatus 52, the input device 53, the communication device 54, and the display device 55 are connected to each other via a bus in such a manner that they can mutually communicate.

The processing circuitry 51 includes processors such as a CPU (central processing unit) and a GPU (graphics processing unit). The processing circuitry 51 realizes an acquisition function 511, a training function 512, and a display control function 513 through execution of a medical learning program. Note that the embodiment is not limited to the case in which the respective functions 511 to 513 are realized by a single processing circuit. Processing circuitry may be composed by combining a plurality of independent processors, and the respective processors may execute programs, thereby realizing the functions 511 to 513. The functions 511 and 513 may be respective modularized program constituting a medical learning program. These programs are stored in the storage apparatus 52.

The storage apparatus 52 is a ROM (read only memory), a RAM (random access memory), an HDD (hard disk drive), an SSD (solid state drive), or an integrated circuit storage device, etc. storing various types of information. The storage apparatus 52 may not only be the above-listed storage apparatuses but also be a driver that writes and reads various types of information to and from, for example, a portable storage medium such as a compact disc (CD), a digital versatile disc (DVD), a flash memory, or a semiconductor memory. The storage apparatus 52 may be provided in another computer connected via a network.

The input device 53 accepts various kinds of input operations from an operator, converts the accepted input operations to electric signals, and outputs the electric signals to the processing circuitry 51. Specifically, the input device 53 is connected to an input device, such as a mouse, a keyboard, a trackball, a switch, a button, a joystick, a touch pad, or a touch panel display. The input device 53 outputs to the processing circuitry 51 an electrical signal corresponding to an input operation on the input device. An audio input apparatus may be used as an input device 53. The input device 53 may be an input device provided in another computer connected via a network or the like.

The communication device 54 is an interface for sending and receiving various types of information to and from other computers. An information communication by the communication device 54 is performed in accordance with a standard suitable for medical information communication, such as DICOM (digital imaging and communications in medicine).

The display device 55 displays various types of information in accordance with the display control function 513 of the processing circuitry 51. For the display device 55, for example, a liquid crystal display (LCD), a cathode ray tube (CRT) display, an organic electro luminescence display (OELD), a plasma display, or any other display can be used as appropriate. A projector may be used as the display device 55.

Through realization of the acquisition function 511, the processing circuitry 51 acquires a data set that consists of a plurality of medical events and that includes a first data sample that includes first action data relating to an expert. An “expert” means a medical care provider who has a high medical care skill (skilled person). An expert in the present embodiment is not limited to a person who is qualified or certified as an expert, and includes a person who is assumed to be relatively adept in comparison to an average person. The first data sample may include first attribute data corresponding to first action data, as mentioned above. The data set may further include a second data sample that includes second action data relating to a non-expert. The second data sample may include second attribute data corresponding to second action data, similarly to the first data sample. A “non-expert” is a person whose medical skill is not high. The non-expert is not limited to a medical care provider and may be any person. A non-expert is not limited to a person who is qualified or certified as a non-expert but also a person who is assumed to be relatively not skillful in comparison to an average person.

Through realization of the training function 512, the processing circuitry 51 trains a causal structural model for inferring a causal relationship relating to a plurality of medical events based on the first data sample acquired by the acquisition function 511. The trained causal structure model is stored in the AI model storing apparatus 7.

Through realization of the training function 512, the processing circuitry 51 trains the causal structure model based on an evaluation function relating to near-optimality relating to the first action data. The evaluation function relating to near-optimality may include a first evaluation function regarding a difference between the first action data and the second action data. For example, the processing circuitry 51 updates parameters of a causal structure model so that the first evaluation function is maximized. The evaluation function relating to near-optimality may include a second evaluation function regarding a reward given to the first action data. The processing circuitry 51 then updates parameters of the causal structure model based on the second evaluation function. For example, the processing circuitry 51 updates parameters of a causal structure model so that the second evaluation function is maximized. The second evaluation function may further include a reward distribution. In this case, the processing circuitry 51 sets a reward distribution as a target distribution and updates parameters of the causal structure model in such a manner that a reward distribution obtained through a first action gets close to the target distribution, in other words, a difference between the reward distribution and the target distribution becomes small. The reward is determined based on a reward function. The reward function is trained through inverse reinforcement learning, for example.

The first action data includes data generated based on a policy function of an expert. The second action data includes data generated based on a policy function of a non-expert. The policy function of an expert is trained through reinforcement learning or imitation learning. The policy function of a non-expert is trained through reinforcement learning or imitation learning.

The data set may include a third data sample generated by a world model. The causal structure model is an example of a world model.

The processing circuitry 51 may train the causal structure model based on an evaluation function relating to causal identifiability conditions. The evaluation function relating to causal identifiability conditions includes at least one of a regression error of data generated from a causal structure, restrictive conditions for generating a directed acyclic graph, or a regularization term relating to complexity of a graph structure or a neural network. The evaluation function relating to causal identifiability conditions may be at least one of a conditional reference or an information criterion.

The processing circuitry 51 may train the causal structure model for inferring a causal relationship relating to a medical event, except for the first action data included in the first data sample. For example, the processing circuitry 51 may train a causal structure model for inferring a causal relationship relating to first attribute data included in a first data sample.

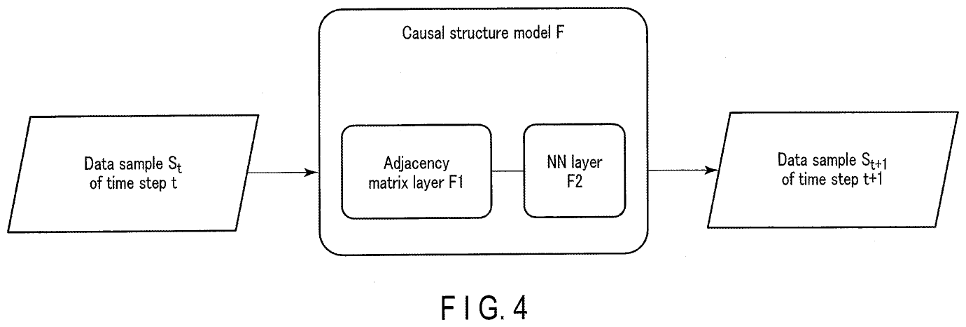

FIG. 4 is a diagram showing an example of a network structure of a causal structure model F. As shown in FIG. 4, the causal structure model F generates a most-likely data sample as a data sample Sₜ₊₁ of a next time step t+1 from a data sample Sₜ of a time step t in view of a causal relationship between multiple medical events. A data sample that seems probable in view of a causal relationship between multiple medical events is generated as a data sample Sₜ₊₁. A causal relationship between medical events is typically from action to attribute or from one attribute to another, but this does not exclude causal relationship from attribute to action or from one action to another. The causal structure model F has an adjacency matrix layer F1 and a neural network (NN) layer F2.

The adjacency matrix layer F1 is a network layer that applied an adjacency matrix A to a data sample Sₜ of a processing-targeted time step t. The adjacency matrix A defines a presence/absence of a causal structure between predetermined multiple medical events. In other words, the adjacency matrix layer F1 infers a medical event that has a causal relationship with a medical event represented by a data sample Sₜ of a time step t. The adjacency matrix layer F1 outputs a data sample S′ₜ to which the adjacency matrix A is applied. The adjacency matrix layer F1 is expressed by a graphical model representing a causal structure between predetermined multiple medical events. A graphical model is defined by a skeleton, a directed graph, a partially directed acyclic graph, a directed acyclic graph, or a topological order.

As an example, suppose the graphical model is a directed acyclic graph constituted by a plurality of nodes respectively corresponding to the predetermined medical events, and an edge representing a causal structure between adjacent nodes (medical events). A variable indicating an attribute and/or an action corresponding to a medical event is allocated to each node. Each node may be called a “medical event variable”. A presence/absence of a causal structure relating to each combination of all nodes included in the graphical model is represented by the adjacency matrix A. The adjacency matrix A has the number of elements corresponding to a combination of nodes (medical event nodes) (hereinafter, an adjacency matrix element). For example, if a causal structure between nodes is present, the adjacency matrix element corresponding to this node combination has the value “1”, and if there is no causal structure between nodes, the adjacency matrix element corresponding to this node combination has the value “0”. The adjacency matrix element is an example of a parameter of a causal structure model F trained by the training function 512.

The NN layer F2 is a network layer for inferring a data sample Sₜ₊₁ of a next time step t+1 based on a data sample S′ₜ to which the adjacency matrix A is applied. The NN layer F2 is constituted by a combination of discretionarily selected network layers, such as a convolutional layer, a fully connected layer, a pooling layer, a regularization layer, and an output layer. Examples of a parameter of the causal structure F trained by the training function 512 are a weight parameter of the NN layer F2 and a network parameter such as a bias.

Through realization of the display control function 513, the processing circuitry 51 causes the display device 55 to display various information items. As an example, the processing circuitry 51 may cause a data sample or a data set to be displayed. As another example, the processing circuitry 51 may cause a result of training a medical structure model or the like to be displayed.

Hereinafter, the medical learning processing by the medical learning apparatus 500 according to the present embodiment is described.

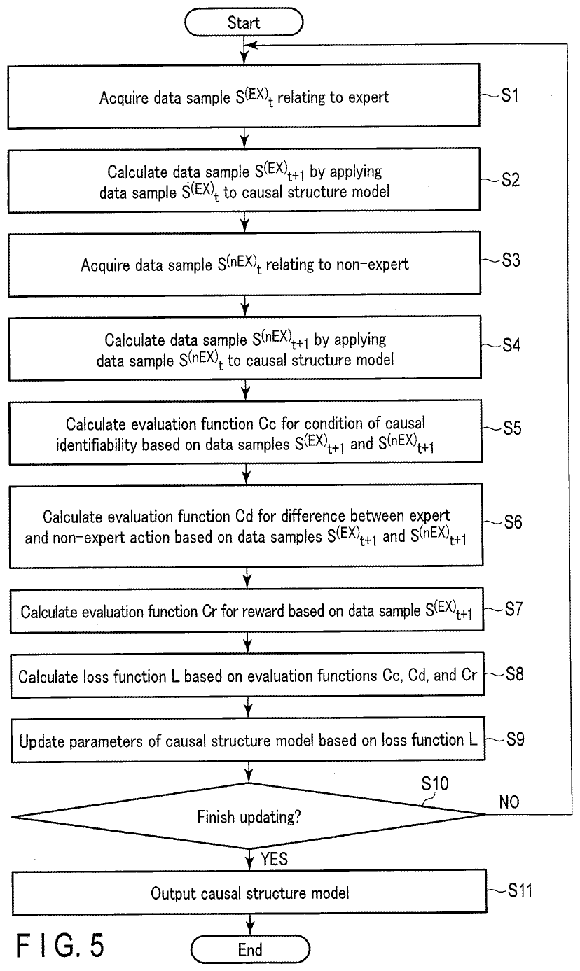

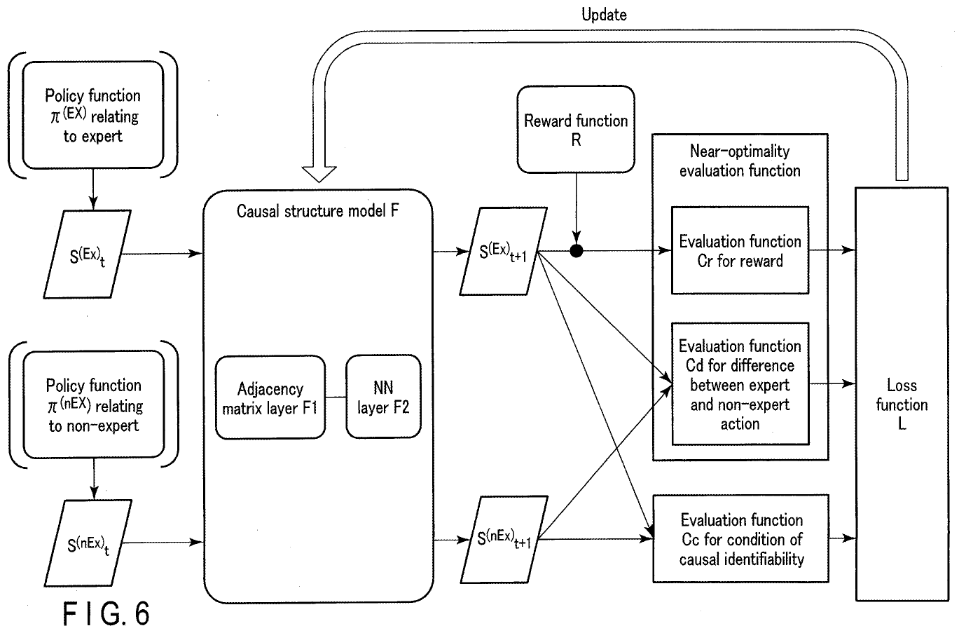

FIG. 5 is a diagram showing an example of procedures of medical learning processing. FIG. 6 is a drawing schematically showing the medical learning processing shown in FIG. 5.

As shown in FIG. 5, the processing circuitry 51 acquires a data sample S^{(EX)}ₜ of a current time step t relating to an expert through realization of the acquisition function 511 (step S1). The data sample S^{(EX)}ₜ may be a factual data sample collected by the medical event collecting apparatus 1 or a counterfactual data sample generated by a policy function π^{(EX)} relating to an expert.

The policy function π^{(EX)} relating to an expert is a model trained to imitate an action of an expert. The policy function π^{(EX)} infers action data of an action that an expert would take from attribute data of a data sample relating to the expert. It is preferable that the policy function π^{(EX)} be trained through reinforcement learning or imitation learning based on a data set of attribute data and action data relating to an expert. As imitation learning, action cloning, GAIL (generative adversarial imitation learning), or apprenticeship learning in which reinforcement learning and inverse reinforcement learning are combined may be adopted.

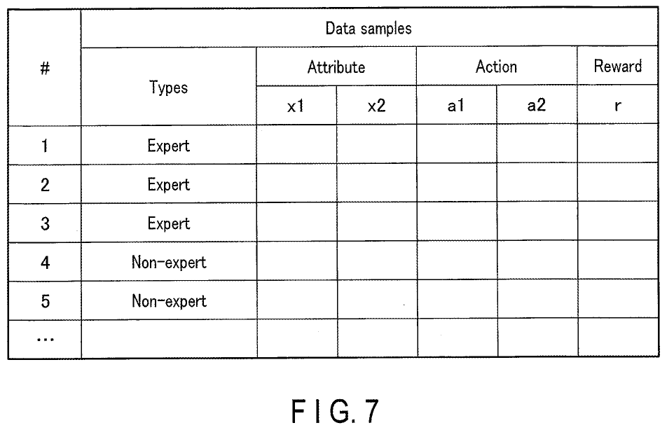

FIG. 7 is a diagram showing a data structure of a data sample according to the medical learning processing shown in FIG. 5. As shown in FIG. 7, a data sample relating to a medical event include an attribute x, an action a, and/or a reward r. Each data sample is associated with an identifier representing a type of a subject of action data included in the data sample. Specifically, types of a subject are an expert or a non-expert.

After step S1, the processing circuitry 51 applies, through realization of the training function 512, the data sample S^{(EX)}ₜ acquired in step S1 to the causal structure model F, and calculates the data sample S^{(EX)}ₜ₊₁ of the time step t+1 (step S2). The causal structure model F used in step S2 is a trainable machine learning model with which training of parameters has not yet been completed.

After step S2, the processing circuitry 51 acquires, through the realization of the acquisition function 511, a data sample S^{(nEX)}ₜ of the current time step t relating to a non-expert (step S3). The data sample S^{(nEX)}ₜ may be a factual data sample stored in the medical event storing apparatus 3 or a counterfactual data sample generated by a policy function π^{(nEX)} relating to a non-expert.

The policy function π^{(nEX)} relating to a non-expert is a model trained to clone an action of a non-expert. The policy function π^{(nEX)} infers, from attribute data of a data sample relating to the expert, action data of an action that a non-expert would take. It is preferable that the policy function π^{(nEX)} be trained through reinforcement learning or imitation learning based on a data set of attribute data and action data relating to an expert. As imitation learning, action cloning, GAIL, apprenticeship learning, etc. may be adopted.

After step S3, the processing circuitry 51 applies, through realization of the training function 512, the data sample S^{(nEX)}ₜ acquired in step S3 to the causal structure model F, and calculates the data sample S^{(nEX)}ₜ of the time step t+1 (step S4). The causal structure model F used in step S4 is the same as the machine learning model used in step S2 and is a trainable machine learning model with which training of parameters has not yet been completed.

After step S4, the processing circuitry 51 calculates, through realization of the training function 512, a causal identifiability condition evaluation function Cc based on the data sample S^{(EX)}ₜ₊₁ calculated in step S2 and the data sample S^{(nEX)}ₜ₊₁ calculated in step S4 (step S5). The causal identifiability condition evaluation function Cc is an evaluation function necessary to specify a correct causal structure from a data sample. For example, when causal discovery is performed as a continuous optimization problem, the causal identifiability condition evaluation function Cc is designed based on a regression error of data generated from a causal structure, restrictive conditions to make a graph a DAG, and a regularization term related to a complexity of a graph structure or a neural network, and the like. As another example, when a causal discovery is performed as a combination optimization problem, the causal identifiability condition evaluation function Cc is designed based on conditioned independence and information criterion, etc. In the causal discovery according to the present embodiment, a DAG is not a prerequisite causal structure.

After step S5, the processing circuitry 51 calculates, through realization of the training function 512, an action difference evaluation function Cd of an expert and a non-expert based on the data sample S^{(EX)}ₜ₊₁ calculated in step S2 and the data sample S^{(nEX)}ₜ₊₁ calculated in step S4 (step S6). The action difference evaluation function Cd is a function for evaluating a difference between action data included in the data sample S^{(EX)}ₜ₊₁ and action data included in the data sample S^{(nEX)}ₜ₊₁.

After step S6, the processing circuitry 51 calculates, through realization of the training function 512, a causal identifiability condition evaluation function Cr based on the data sample S^{(EX)}ₜ₊₁ calculated in step S2 (step S7). The reward evaluation function Cr is a function for evaluating reward data given to the action data included in the data sample S^{(EX)}ₜ₊₁. The reward data may be artificially generated or generated based on a reward function R.

The reward function R is a model trained to infer reward data from the attribute data and the action data included in the data sample S^{(EX)}ₜ₊₁. It is preferable that the reward function R be trained through inverse reinforcement learning based on a data set of attribute data and action data relating to an expert.

After step S7, the processing circuitry 51 calculates, through realization of the training function 512, a loss function L based on the evaluation function Cc calculated in step S5, the evaluation function Cd calculated in step S6, and the evaluation function Cr calculated in step S7 (step S8). The loss function L is formulated by weighted addition of the evaluation functions Cc, Cd, and Cr, as shown in Expression (1) below. The ratio between the weights wc, wd, and wr are adjustable at a user's discretion.

L=wc·Cc+wd·Cd+wr·Cr (1)

The evaluation functions Cd and Cr are evaluation functions relating to near-optimality of action data of an expert. Near-optimality means that the action data of an expert is optimal or almost optimal. As described earlier, the evaluation function Cd evaluates action data included in the data sample S^{(EX)}ₜ₊₁ and action data included in the data sample S^{(nEX)}ₜ₊₁. More specifically, the evaluation function Cd is a function for evaluating a distance between a feature amount obtained from the data sample S^{(EX)}ₜ₊₁ of an expert and a feature amount of the data sample S^{(nEX)}ₜ₊₁ of a non-expert. As an example, an evaluation function Cd may be designed in such a manner that a value of the evaluation function Cd becomes smaller as the distance becomes larger. In this case, if the action data of an expert has near-optimality, the value of the evaluation function Cd becomes relatively small as the distance becomes relatively large. As described earlier, the evaluation function Cr evaluates reward data given to action data included in the data sample S^{(EX)}ₜ₊₁. As an example, the evaluation function Cr may be designed in such a manner that a value of the evaluation function Cr with a higher reward becomes small. In this case, if the action data of an expert has near-optimality, the reward becomes relatively high and therefore the value of the evaluation function Cr becomes relatively small.

After step S8, the processing circuitry 51 updates, through realization of the training function 512, the parameters of the causal structure model F based on the loss function L calculated in step S8 (step S9). The processing circuitry 51 updates parameters so as to minimize a value (loss) of the loss function L. More specifically, the processing circuitry 51 updates the parameters so that the evaluation functions Cc, Cd, and Cr are minimized. Regarding the adjacency, the parameters are updated in such a manner that a distance of the feature amount obtained from the data sample S^{(EX)}ₜ₊₁ of an expert and a feature amount obtained from the data sample S^{(nEX)}ₜ₊₁ of a non-expert, which is defined by the evaluation function Cd, is maximized, and a reward given to the action data included in the data sample S^{(EX)}ₜ₊₁ is maximized.

To make a loss smaller as the value of the loss function L becomes larger, it is also possible to design the loss function L by inverting the signs of the evaluation functions Cc, Cd, and Cr. In this case, the processing circuitry 51 may update the parameters to maximize the value (loss) of the loss function L.

After step S9, through the realization of the training function 512, the processing circuitry 51 determines whether or not the condition for finishing updating is satisfied (step S10). The condition for finishing updating may be set to a discretionarily selected condition, such as finishing of a training of a predetermined number of data samples and a performance index of a causal structure model reaching a predetermined criterion, or the like. If it is determined that the condition for finishing updating is not satisfied (No in step S10), the processing circuitry 51 performs steps S1 through S10 once again for another data sample. The processing circuitry 51 repeats steps S1 through S10, changing a data sample, until it is determined that the condition for finishing updating is satisfied in step S10.

If it is determined that the condition for finishing updating is satisfied (Yes in step S10), the processing circuitry 51 outputs a current causal structure model F (step S11). The output causal structure model F may be stored in the storage apparatus 52, stored in the AI model storing apparatus 7, or transferred to the medical inference apparatus 9.

The medical learning process is thus finished.

The order of the procedures of the medical learning processing described in the above and shown in FIGS. 5 and 6 is merely an example and the present embodiment is not limited to this example.

For example, the order of the acquisition of the data sample S^{(EX)}ₜ (S1) and the calculation of the data sample S^{(EX)}ₜ₊₁ (S2) and the acquisition of the data sample S^{(nEX)}ₜ (S3) and the calculation of the data sample S^{(nEX)}ₜ₊₁ (S4) may be inverted, and these steps may be performed in parallel. Furthermore, the calculation of the causal identifiability condition evaluation function Cc (S5), the calculation of the action difference evaluation function Cd (S6), and the calculation of the reward evaluation function Cr (S7) may be performed in any order.

In the above-described medical learning processing, the parameters are updated in such a manner that the loss function L based on the causal identifiability condition evaluation function Cc, the action difference evaluation function Cd, and the reward function Cr is minimized. However, the present embodiment is not limited to this example. It suffices that the parameters are updated based on at least one type of evaluation function Cc, Cd, and Cr. More restrictively, it suffices that the parameters are updated based on the evaluation function Cc and/or the evaluation function Cr, which are the evaluation functions relating to near-optimality. It is thus possible to train the causal structure model F by weighting the action data of an expert in relation to the action data of a non-expert.

According to the foregoing description, the medical learning apparatus 5 according to the present embodiment has processing circuitry 51. The processing circuitry 51 acquires a data set consisting of a plurality of medical events and including a first data sample that includes first action data relating to an expert. The processing circuitry 51 trains, based on the first data sample, a causal structure model for inferring a causal structure relating to a plurality of medical events.

According to the above configuration, the causal structure model is trained using a data sample relating to an expert; it is thus possible to improve accuracy of inference of a causal structure of multiple medical events by the causal structure model. In turn, improvement in accuracy in downstream tasks, such as diagnosis of disease, individualized treatment effect prediction, dynamic treatment regimens, etc. through a use of a causal structure model is expected.

### APPLICATION EXAMPLES

In the foregoing embodiment, it is assumed that the causal structure model F, the reward function R, and the policy functions π^{(EX)}, π^{(nEX)} are separately generated. A processing circuitry 51 according to an applied example may be generated in conjunction with the causal structure model F, the reward function R, and the policy functions π^{(EX)}, π^{(nEX)}.

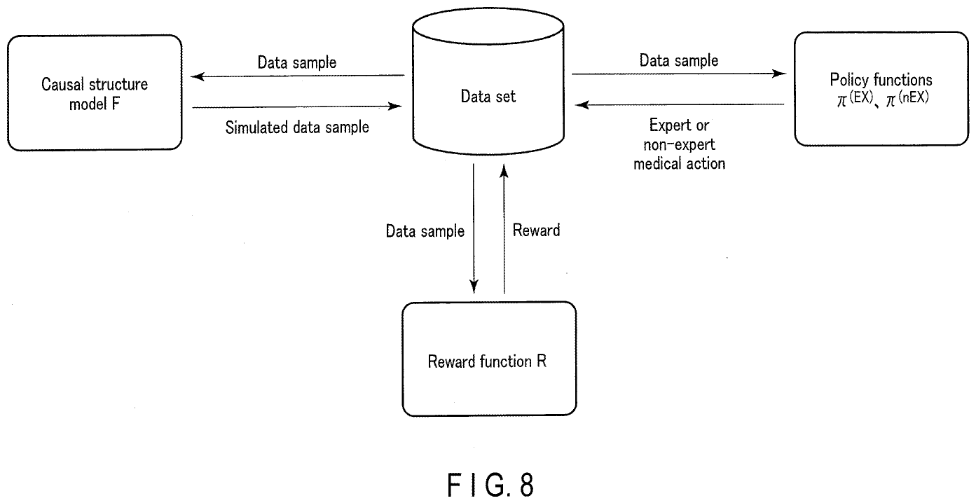

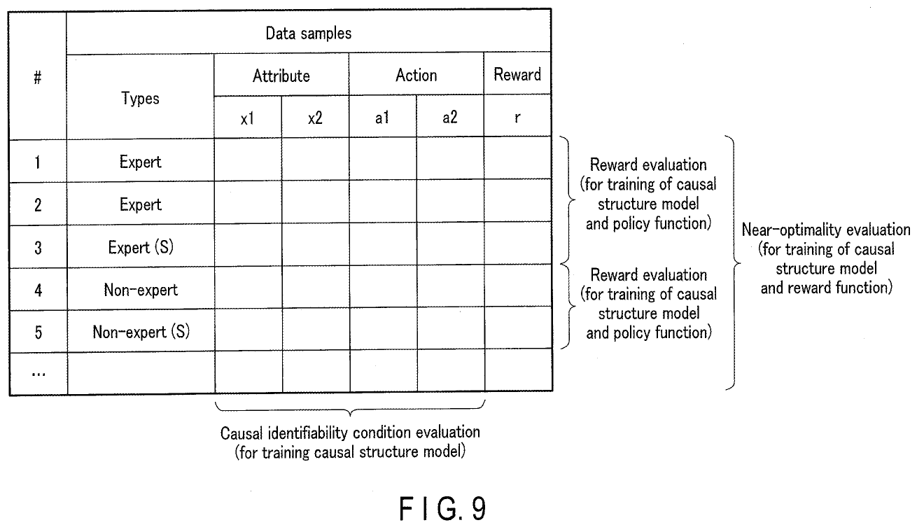

FIG. 8 is a diagram showing receipt and transmission of data between the causal structure model F, the reward function R, and the policy functions π^{(EX)}, π^{(nEX)}. FIG. 9 is a diagram showing a data structure of a data sample according to an applied example. The causal structure model F is, in general, a model representing a data generation process. Herein, the processing circuitry 51 generates counterfactual data samples using the causal structure model F. Specifically, the processing circuitry 51 generates a data sample Sₜ₊₁ of the time step t+1 by applying the data sample Sₜ of the time step t to the causal structure model F. The data sample Sₜ₊₁ acquired using the causal structure model F means a counterfactual data sample, not an actually measured data sample. The data sample Sₜ₊₁ acquired using the causal structure model F may be called a “simulated data sample”. The data sample Sₜ₊₁ acquired using the causal structure model F is either action data or a set of action data and attribute data. Since the time steps t and t+1 will be treated identically in the process hereinafter, the description of the time steps t and t+1 is omitted.

The processing circuitry 51 may generate a simulated data sample S^{(EX)} from the data sample S^{(EX)} relating to an expert or may generate a simulated data sample S^{(nEX)} from the data sample S^{(nEX)} relating to a non-expert. The simulation data samples S^{(EX)} and/or S^{(nEX)} are added to a data set in the medical event storing apparatus 3. At this time, as shown in FIG. 9, the factual data sample (actually measured data sample) and the counterfactual data sample (simulated data sample) are stored in an identifiable manner. In the example shown in FIG. 9, “(S)” is given to a counterfactual data sample.

The processing circuitry 51 generates action data by applying a policy function to a factual and/or counterfactual data sample. Specifically, the processing circuitry 51 generates action data by applying the policy function π^{(EX)} to the factual and/or counterfactual data sample relating to an expert. Similarly, the processing circuitry 51 generates action data by applying the policy function π^{(nEX)} to the factual and/or counterfactual data sample relating to a non-expert. The action data acquired through a use of the policy function is expected to have a higher accuracy compared to the action data acquired through a use of the causal structure model. The processing circuitry 51 writes over the action data acquired through a causal structure model with the action data acquired through using the policy function.

The processing circuitry 51 generates reward data relating to the data sample by applying the reward function to the factual and/or counterfactual data sample after the action data is overwritten. The generated reward data is allocated to the data sample. The data sample is thus completed.

The causal structure model that uses the evaluation function Cc of the causal identifiability conditions is trained based on the attribute data and/or the action data of the data samples relating to an expert and a non-expert. The causal structure model F and the policy function π^{(EX)} are trained with a reward maximization method using the reward evaluation function Cr based on the data sample relating to an expert. The causal structure model F and the policy function π^{(nEX)} are trained with a reward maximization method using the reward evaluation function Cr based on the data sample relating to a non-expert. The causal structure model F and the reward function R are trained through an adverse learning method using an evaluation function Cd and/or Cc relating to near-optimality based on a data sample relating to an expert and a non-expert. In the training of a causal structure model F, a policy function n, and a reward function R, any one of the models may be fixed and the rest of the models may be trained, or all of the models may be trained at the same time.

According to the applied example, it is possible to train a causal structure model F, a policy function n, and a reward function R effectively and with high accuracy through amplifying a highly accurate data sample by the causal structure model F, the policy function n, and the reward function R.

According to at least one of the foregoing embodiments, it is possible to accurately infer a causal structure relating to a medical event.

The term “processor” used in the above explanation indicates, for example, a circuit, such as a CPU, a GPU, or an Application Specific Integrated Circuit (ASIC), and a programmable logic device (for example, a Simple Programmable Logic Device (SPLD), a Complex Programmable Logic Device (CPLD), and a Field Programmable Gate Array (FPGA)). The processor realizes its function by reading and executing the program stored in the storage circuitry. The program may be directly incorporated into the circuit of the processor instead of being stored in the storage circuit. In this case, the processor implements the function by reading and executing the program incorporated into the circuit. If the processor is for example an ASIC, on the other hand, the function is directly implemented in a circuit of the processor as a logic circuit, instead of storing a program in a storage circuit. Each processor of the present embodiment is not limited to a case where each processor is configured as a single circuit; a plurality of independent circuits may be combined into one processor to realize the function of the processor. In addition, a plurality of structural elements in FIG. 1 may be integrated into one processor to realize the function.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical learning apparatus comprising processing circuitry configured to:
acquire a data set consisting of a plurality of events, the data set including first data that includes first action data relating to an expert; and
train, based on the first data, a causal structure model for inferring a causal relationship relating to the plurality of events.

2. The medical learning apparatus of claim 1, wherein
the first data sample includes first attribute data corresponding to the first action data.

3. The medical learning apparatus of claim 1, wherein
the data set further includes a second data sample that includes second action data relating to a non-expert.

4. The medical learning apparatus of claim 3, wherein
the second data sample includes second attribute data corresponding to the second action data.

5. The medical learning apparatus of claim 1, wherein
the processing circuitry trains the causal structure model based on an evaluation function relating to near-optimality relating to the first action data.

6. The medical learning apparatus of claim 5, wherein
the data set further includes a second data sample that includes second action data relating to a non-expert,
the evaluation function is a first evaluation function relating to a difference between the first action data and the second action data, and
the processing circuitry updates a parameter of the causal structure model in such a manner that the first evaluation function is maximized.

7. The medical learning apparatus of claim 5, wherein
the evaluation function is a second evaluation function relating to a reward given to the first action data, and
the processing circuitry updates a parameter of the causal structure model based on the second evaluation function.

8. The medical learning apparatus of claim 7, wherein
the processing circuitry updates a parameter of the causal structure model in such a manner that the second evaluation function is maximized.

9. The medical learning apparatus of claim 7, wherein
the second evaluation function further includes a distribution of the reward, and
the processing circuitry updates the causal structure model in such a manner that a difference between the distribution of the reward and a target distribution of a reward becomes small.

10. The medical learning apparatus of claim 7, wherein
the reward is determined based on a reward function.

11. The medical learning apparatus of claim 10, wherein
the reward function is trained by inverse reinforcement learning.

12. The medical learning apparatus of claim 1, wherein
the first action data includes data generated based on a policy function of an expert.

13. The medical learning apparatus of claim 3, wherein
the second action data includes data generated based on a policy function of a non-expert.

14. The medical learning apparatus of claim 12, wherein
the policy function of an expert is trained through reinforcement learning or imitation learning.

15. The medical learning apparatus of claim 13, wherein
the policy function of a non-expert is trained through reinforcement learning or imitation learning.

16. The medical learning apparatus of claim 1, wherein
the data set includes a third data set generated by a world model.

17. The medical learning apparatus of claim 1, wherein
the processing circuitry trains the causal structure model further based on an evaluation function relating to causal identifiability conditions.

18. The medical learning apparatus of claim 17, wherein
the evaluation function relating to the causal identifiability conditions is at least one of a regression error of data generated from a causal structure, restriction conditions for generating a directed acyclic graph, and a regularization term relating to a complexity of a graph structure or a neural network.

19. The medical learning apparatus of claim 17, wherein
the evaluation function relating to the causal identifiability conditions is at least one of a conditional reference or an information criterion.

20. The medical learning apparatus of claim 1, wherein
the causal structure model is a world model.

21. The medical learning apparatus of claim 1, wherein
the processing circuitry trains a causal structure model for inferring a causal relationship relating to an event, except for the first action data included in the first data sample.

22. The medical learning apparatus of claim 2, wherein
the processing circuitry trains a causal structure model for inferring a causal relationship relating to the first attribute data.

23. The medical learning apparatus of claim 1, wherein
the causal structure model is at least one of a skeleton, a directed graph, a partially directed acyclic graph, a directed acyclic graph, or a topological order.

24. A medical information processing method comprising:
a step of acquiring a data set consisting of a plurality of events, a data set including a first data sample that includes first action data relating to an expert; and
a step of training, based on the first data, a causal structure model for inferring a causal relationship relating to the plurality of events.

25. A medical information processing system comprising:
a collection apparatus configured to collect a data set consisting of a plurality of events, the data set including a first data sample that includes first action data relating to an expert;
a training apparatus configured to train, based on the first data, a causal structure model for inferring a causal relationship relating to the plurality of events; and
an inference apparatus for inferring a data sample of a time step at a next point of time from a data sample of a time step at a current point of time.
